(19) United States
(12) Patent Application Publication
(10) Pub. No.: US 2012/0028286 A1
(43) Pub. Date: Feb. 2, 2012

(54) METHOD FOR EVALUATING THE BREAKDOWN OF PROTEINS, POLYPEPTIDES AND PEPTIDES

(76) Inventor: Charles F. Saller, Escondido, CA (US)

(21) Appl. No.: 13/195,413

(22) Filed: Aug. 1, 2011

Related U.S. Application Data
(60) Provisional application No. 61/369,304, filed on Jul. 30, 2010.

(51) Int. Cl. C12Q 1/37; C12Q 1/00

(52) U.S. Cl. 435/23; 435/40.5

(57) ABSTRACT

Provided is an assay for determining the protein, polypeptide, and peptides degradation of a sample. The assay for determining the protein, polypeptide, and peptides degradation of a sample includes contacting the sample with a non-fluorescent compound whereby the non-fluorescent compound reacts with free primary amine in the sample from degradation of protein, polypeptide, and peptides in the sample producing a fluorescent signal to thereby measure the increase in protein, polypeptide, and peptides fragments in the sample and comparing the fluorescent signal to a standard to thereby quantify the amount of protein, polypeptide, and peptides degradation. By taking the ratio of this increase to protein concentrations measured using standard protein assays a sensitive measure of protein degradation is obtained.

### CROSS REFERENCE TO RELATED APPLICATION

This application claims priority under 35 USC 119(e) from U.S. provisional application Ser. No. 61/369,304 to Saller, filed Jul. 30, 2010, entitled Method for Evaluating the Breakdown of Proteins, entire disclosure of which is incorporated herein by reference.

### TECHNICAL FIELD

The present disclosure relates to a method for evaluating the breakdown of proteins, polypeptides, and peptides in a sample. In particular, the assay of the present disclosure measures the increase in the protein, polypeptide and/or peptide fragments as a measure of protein, polypeptide and/or peptide degradation.

### BACKGROUND OF DISCLOSURE

Proteins, polypeptides, and peptides degrade in biological samples and in solution. For example, following the cessation of blood flow, proteases are activated resulting in protein breakdown resulting in an increase in the number of protein and peptide fragments, including free amino acids. Determining protein degradation of particular proteins would be helpful and desirable.

### SUMMARY OF DISCLOSURE

The present disclosure relates to an assay that measures the increase in the number of protein, polypeptide, and peptide fragments and free amino acids in a sample as a measure of protein, polypeptide, and peptides degradation. In particular, the present disclosure relates to an assay for determining the protein, polypeptide, and peptides degradation of a sample which comprises contacting the sample with a non-fluorescent compound whereby the non-fluorescent compound reacts with free primary amine in the sample from degradation of protein, polypeptides, and peptides in the sample producing a fluorescent signal to thereby quantify the increase in protein, polypeptide and/or peptide fragments in the sample and comparing the fluorescent signal to a standard to thereby quantify the amount of protein, polypeptide, and peptides degradation. Examples of non-fluorescent compounds that can be utilized as fluorescent tags for free amines according to the present disclosure are 3-(4-carboxybenzoyl) quinoline-2-carboxaldehyde (CBQCA), fluorescamine and o-phthaldialdehyde.

The present disclosure also relates to an assay that utilizes both the assay employing the fluorescent tag for free amines, (e.g., 3-(4-carboxybenzoyl) quinoline-2-carboxaldehyde ((CBQCA)), fluorescamine and o-phthaldialdehyde) and a common protein assay, of which the signal does not change or decrease as protein are degraded.

The commonly used protein assays such as the Bradford, Lowry, Pierce 660 nm (Thermo Fisher Scientific Inc.), DC (Bio-Rad Laboratories), and bicinchoninic acid (BCA) protein assays rely upon the detection of either peptide bonds or specific amino acids in proteins or polypeptides but these assays do not detect free amines or most free amino acids. Therefore, the signal for these commonly used protein assays either does not change or may decrease if a significant number of peptide bonds are broken. The output from these is measured as a change of absorbance determined by a spectrophotometer or microplate reader. This output can serve a baseline measure of protein content; but as noted, may decrease if the sample is highly degraded.

Because the signal from the abovementioned commonly used protein assays may decrease due to protein or polypeptide degradation, by measuring the ratio of the fluorescence increase due to CBQCA or other compounds that fluoresce upon reaction with free amines to the signal of commonly used protein assays, the sensitivity of this Protein Degradation Assay may be increased. More specifically, the numerator of this ratio is the fluorescence signal output of the CBQCA and similarly reacting compounds and the denominator is the signal of any one the commonly used protein assays. Because the numerator increases as function of protein degradation and the denominator is either stable or decreases, depending upon the extent of protein degradation the ratio will increase as proteins or polypeptides are increasingly broken down.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF DRAWING

FIG. 1A is a calibration curve showing the labeling of a BSA protein standard and amino acids with CBQCA. FIG. 1B is a calibration curve showing the labeling of a BSA protein standard, but representative free amino acids are not detected in the Pierce 660 nm protein assay.

FIG. 2 demonstrates that the incubation of protein lysate at elevated temperature leads to increased labeling by CBQCA, indicating protein degradation, and a small decline in protein labeling by the commonly used Bradford protein assay.

FIG. 3 demonstrates that CBQCA detects fragmentation of proteins due to increasing duration of trypsin digestion.

FIG. 4 demonstrates that CBQCA detects increases in protein digestion but the 660 nm protein assay shows a decrease in protein concentration as increased amounts of trypsin are present.

FIG. 5 shows the CBQCA/660 nm Protein Degradation Assay ratio increase as additional trypsin digestion occurs.

### BEST AND VARIOUS MODES FOR CARRYING OUT DISCLOSURE

The present disclosure provides an assay to measure the increase in protein, polypeptide, or peptide fragments in a sample as a measure of protein, polypeptide, and peptide degradation. The assay of the present disclosure for determining the protein, polypeptide and/or peptide degradation of a sample comprises contacting the sample with a non-fluorescent compound whereby the non-fluorescent compound reacts with free primary amine in the sample from degradation of proteins, polypeptides, and peptides in the sample producing a fluorescent signal to thereby quantify the increase in protein, polypeptide or peptide fragments in the sample and comparing the fluorescence to a standard to thereby determine the extent of protein degradation. Examples of non-fluorescent compounds that become fluorescent after reaction with free amines according to the present disclosure are 3-(4-carboxybenzoyl) quinoline-2-carboxaldehyde (CBQCA); fluorescamine and o-phthaldialdehyde.

CBQCA does not fluoresce appreciably in aqueous solution. However, in the presence of cyanide, it reacts with primary amines to form highly fluorescent derivatives. Please see Liu J P, Hsieh Y Z, Wiesler D, Novotny M., Design of 3-(4-carboxybenzoyl)-2-quinolinecarboxaldehyde as a reagent for ultrasensitive determination of primary amines by capillary electrophoresis using laser fluorescence detection. Anal Chem. 1991 Mar. 1;63(5):408-12. As proteins, polypeptides, and peptides are degraded, the number of primary amines exposed increase with the cleavage of each amino bond: the signal produced by CBQCA should also increase correspondingly. Likewise, individual free amino acids may be released exposing additional free amines. To evaluate this, rat brain samples were frozen on dry ice within 60 seconds of removal or were allowed to sit at ambient temperature (˜22° C.) for 16 hours and then frozen. All samples were homogenized in 0.1 M borate buffer, pH 9.3 and labeled with CBQCA in the presence of cyanide. (CBQCA Protein Quantitation Kit (C-6667) Instructions, Molecular Probes, Inc. Eugene, Oreg.)

The samples that were permitted to degrade at 22° C. exhibited a 50% increase in CBQCA labeling (P<0.5), suggesting that this method may be useful in providing a rapid measure of protein breakdown.

In a similar experiment, protein concentrations were measured using the Bradford protein assay. With samples frozen immediately and those assayed after 16 hours at 22° C., protein concentrations were not significantly different. The Bradford assay utilizes the dye Coomassie Blue G, which labels arginine and to a lesser extent histidine, lysine, and aromatic amino acids in proteins. Over this time period, these amino acids are likely intact and therefore still available for labeling; whereas, as peptide bonds are cleaved more amine groups are exposed and the CBCQA labeling is increased. These data indicate that CBCQA can be used to monitor protein breakdown and the Bradford assay can still provide a measure of sample protein concentration.

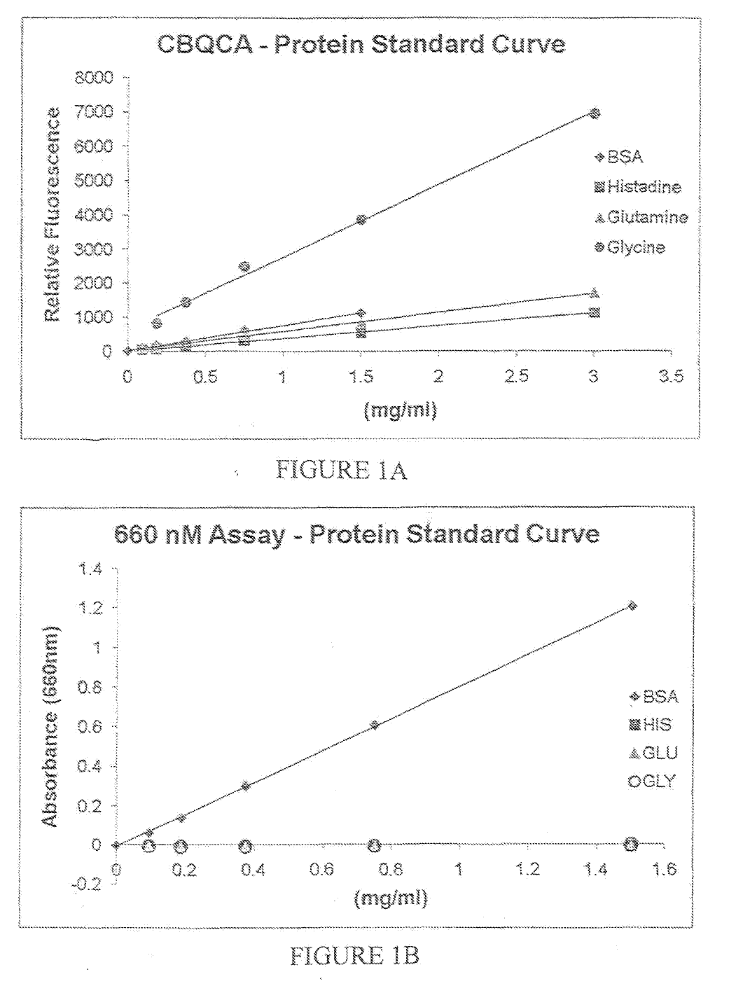

FIG. 1A is a calibration curve showing the labeling of a BSA (bovine serum albumin) protein standard with CBQCA. The fluorescence emission was measured at 550 nm, excitation at 465 nm. It also shows that various free amino acids that can result from protein, polypeptide, and peptide degradation are detected by the method.

Fluorescamine reacts with primary amine groups on proteins; and the unbound dye is non-fluorescent. OPA (o-phthaldialdehyde) reacts with primary amine groups on proteins and amino acids in the presence of 2-mercaptoethanol and other reducing agents; and the unbound dye is non-fluorescent. Therefore, fluorescamine, OPA, and similarly reacting compounds can be used in the same manner as CBQCA to monitor protein, polypeptide, and peptide degradation.

FIG. 1B shows a standard curve for the Pierce 660 nm protein assay using BSA as the standard. FIG. 1B also demonstrates that representative free amino acids (histidine, glutamine, and glycine) are not detected by this assay.

The following non-limiting examples are presented to further facilitate an understanding of the present invention.

### EXAMPLE 1

Human pancreas tissue that had been previously freezer milled was weighed while keeping the sample on dry ice and placed in a microcentrifuge tube. The tissue was suspended with 10 volumes (tissue weight/volume) H₂O pH 7.0. The samples were then sonicated. Samples were centrifuged in a Sorvall SS34 rotor using adaptors for 1.5 ml tubes at 14,500 RPM for 6 minutes. Upon completion of the centrifugation, samples were placed on ice. 300 μl aliquots were taken from each tissue and placed in a heat block set at 65° C. 50 μl samples were taken at 80 minutes and placed on ice.

The total protein assay to be used was the Coomassie (Bradford) assay from Thermo Scientific. The Bradford reagent was aliquoted into a 15 ml conical tube and allowed to warm to room temperature. 5 μl of each standard was added to the appropriate wells of a microplate as well as 5 μl of the homogenized tissues. The Bradford reagent was gently mixed and then 250 μl was added to each well containing a sample or the standard. The plates were briefly shaken using the plate reader and then incubated at a room temperature and absorbance was read at 595 nm.

The Protein Degradation Assay was set up using CBQCA. 2 μl of standards or a sample were added to a black clear bottom 384 well plate. 31.8 μl of reaction buffer (0.1 M sodium borate pH 9.3) was added to each well with sample. 1.25 μl of KCN was added to each well and mixed. A fresh 2 mM working stock of CBQCA was made by diluting the 40 mM stock solution 1:20 in 0.1 M sodium borate pH 9.3. 2.5 μl of the aqueous 2 mM working solution was added immediately to each well. The samples were protected from light and mixed at room temperature for 1 hour. The plate was read using a kinetic read at a 550 nm emission with an excitation of 465 nm using SoftMax Pro software on a Gemini EM instrument for 30 minutes.

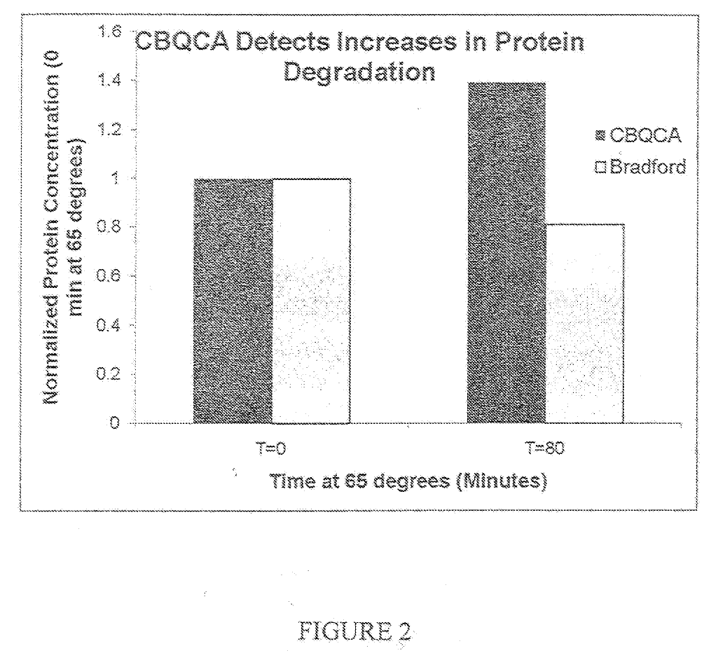

The results of this experiment, shown in FIG. 2, indicated that under the conditions of this experiment, the CBQCA detects an increase in protein degradation, whereas the Bradford protein assay shows a small decline in protein concentrations.

### EXAMPLE 2

Aliquots of freezer milled human lung samples were weighed and suspended in 200 mM NaCl, 0.2% Triton pH 7.0. The samples were vortexed and sonicated. All samples were centrifuged in the SS34 rotor at 3000 RPM for 6 min. Upon completion of centrifugation, samples were placed on ice.

Trypsin was freshly prepared by adding 5.1 mg trypsin to 5.1 ml of 0.1 M sodium borate pH 9.3. 110 μl of the tissue lysates was added in duplicate to a PCR strip. 4.4 μl of 0.1 M sodium borate pH 9.3 was added to the lysate in the first well of the strip. 4.4 μl of trypsin was added to the one well of lysate and samples were placed at 37° C. for 2 hours. Samples were removed from 37° C. and kept on ice while setting up protein assays.

The Protein Degradation Assay was set up using CBQCA. To set up this assay, 31.8 μl of reaction buffer (0.1 M sodium borate pH 9.3) was added to each well of a black clear bottom 384 well plate. 2 μl of standards or sample were added to the well containing 0.1 M sodium borate. 1.25 μl of KCN was added to each well and mixed. A fresh 2 mM working stock of CBQCA was made by diluting the 40 mM stock solution 1:20 in 0.1 M sodium borate pH 9.3. 2.5 μl of the aqueous working solution were added immediately to each well as indicated. The samples were protected from light and mixed at room temperature for 1 hour. The plate was read at a 550 nm emission with an excitation of 465 nm using SoftMax Pro software on a Gemini EM instrument.

Total protein content was assayed using the Pierce 660 nm assay from Thermo Scientific. The 660 nm reagent is stored at room temperature. 10 μl of each standard was added in the indicated wells below as well as 10 μl of the tissue lysates. The room temp Bradford reagent was gently mixed and then 150 μl was added to each indicated well.

The plates were shaken for 1 minute and then incubated at room temperature for 5 minutes. Then the plate was read on the SpectraMax reader using SoftMax Pro software at 660 nm absorbance. Protein concentrations were also measured using the Bio-Rad DC protein assay, according to the manufacturer's instructions.

TABLE 1

| | | DC | Bradford |
|---|---|---|---|
| | | PROTEIN | Protein |
| | CBQCA | ASSAY | Assay |
| −Trypsin (Buffer added) | 1.61 | 1.78 | 1.43 |
| +Trypsin | 1.93 | 1.70 | 0.47 |
| (trypsin concentration | | | |
| subtracted) | | | |

Table 1 shows that CBQCA detects increased protein fragments after trypsin digestion. Protein labeling by the 660 nm Protein Assay is reduced following trypsin digestion of proteins. The ratio of the CBQCA signal to that of the 660 nm Protein Assay is also greatly increased following protein digestion by trypsin relative to samples that were incubated without trypsin. No significant change in protein concentration was observed with the DC Protein Assay.

### EXAMPLE 3

Aliquots of freezer milled human lung samples were weighed and suspended in 200 mM NaCl, 0.2% Triton pH 7.0. The samples were vortexed and sonicated. All samples were centrifuged in a SS34 rotor at 3000 RPM for 6 min. Upon completion of the centrifugation, samples were placed on ice. To prepare the standards for the protein assays, 150 μl of the BSA stock solution from Pierce (2 mg/ml) was serially diluted.

Trypsin was freshly prepared by adding 5.1 mg trypsin to 5.1 ml of 0.1 M sodium borate pH 9.3. 55 μl of the tissue lysates or BSA was added to 6 wells of an 8-well PCR strip. 2.2 μl of 0.1 M sodium borate pH 9.3 was added to the lysate in the first well of each strip. 2.2 μl of trypsin was added to the one well of lysate and samples were placed at 37° C. Every 30 minutes, samples were removed and 2.2 μl of trypsin was added to a new lysate well. Samples were returned to 37° C. between each time point (30, 60, 90, 120 min). Immediately prior to setting up the protein assays, 2.2 μl of trypsin was added to the last lysate well. Samples were kept on ice.

The Protein Degradation Assay was set up using CBQCA. To set up this assay, 31.8 μl of reaction buffer (0.1 M sodium borate pH 9.3) was added to each well of a black clear bottom 384 well plate. 2 82 l of standards or sample were added to the well containing 0.1 M sodium borate. 1.25 μl of KCN was added to each well and mixed well. A fresh 2 mM working stock of CBQCA was made by diluting the 40 mM stock solution 1:20 in 0.1 M sodium borate pH 9.3. 2.5 μl of the aqueous working solution were added immediately to each well as indicated. The samples were protected from light and mixed at room temperature for 1 hour. The plate was read at a 550 nm emission with an excitation of 465 nm using SoftMax Pro software on a Gemini EM instrument.

The total protein assay to be used was the Pierce 660 nm assay from Thermo Scientific. The 660 nm reagent is stored at room temperature. 10 μl of each standard was added in the wells of a microplate as well as 10 μl of the tissue lysates. The Protein Assay reagent was gently mixed and then 150 μl was added to both wells containing standards and samples.

The plates were shaken for 1 minute and then incubated at room temperature for 5 minutes. Then the plates were read on the SpectraMax reader using the SoftMax Pro software at 660 nm absorbance

As can be seen in FIG. 2 and Table 1 above, existing traditional protein assays show no change or decreases in protein concentration due to protein degradation.

### EXAMPLE 4

Freezer milled human lung samples were weighed and suspended in 200 mM NaCl, 0.2% Triton pH 7.0. The samples were vortexed and sonicated. The samples were centrifuged in the SS34 rotor at 3000 RPM for 6 min. Upon completion of the centrifugation, the sample was placed on ice. To prepare the standards for the protein assays, 150 μl of the BSA stock solution from Pierce (2 mg/ml) was place in a microcentrifuge tube containing 50 μl of suspension solution (Final Conc. 1.5 mg/ml).

Trypsin was freshly prepared by adding 6.3 mg trypsin to 6.3 mL of 0.1 M sodium borate pH 9.3. 50 μl of trypsin was added to the first well of an 8-well PCR strip. 10 μl of trypsin was removed from this well and added to 40 μl of 0.1 M sodium borate pH 9.3. This sample was labeled 1:5. The remaining wells had 25 μl of 0.1 M sodium borate pH 9.3 added. 25 μl of the 1:5 diluted trypsin was added to the 25 μl of sodium borate to make a 1:10 dilution. 25 μl of the 1:10 diluted trypsin was added to the 25 μl of sodium borate to make a 1:20 dilution. 25 μl of the 1:20 diluted trypsin was added to the 25 μl of sodium borate to make a 1:40 dilution. 25 μl of the 1:40 diluted trypsin was added to the 25 μl of sodium borate to make a 1:80 dilution. 25 μl of the 1:80 diluted trypsin was added to the 25 μl of sodium borate to make a 1:160 dilution.

50 μl of the tissue lysate was added into wells of 2 8-well PCR strip. In the first strip, 2 μl of trypsin was added from the corresponding trypsin dilution strip. Both lysate strips were then placed at 37° C. for a 2 hour preincubation. Immediately prior to setting up the protein assays, 2 μl of trypsin from the dilution strip was added to each of the wells in the lysate strip that had not yet received trypsin. Samples were kept on ice.

The Protein Degradation Assay was set up using CBQCA. To set up this assay, 31.8 μl of reaction buffer (0.1 M sodium borate pH 9.3) was added to each well of a black clear bottom 384 well plate. 2 μl of standards or sample were added to the well containing 0.1 M sodium borate. 1.25 μl of KCN was added to each well and mixed well. A fresh 2 mM working stock of CBQCA was made by diluting the 40 mM stock solution 1:20 in 0.1 M sodium borate pH 9.3. 2.5 μl of the aqueous working solution were added immediately to each well. The samples were protected from light and mixed at room temperature for 1 hour. The plate was read at a 550 nm emission with an excitation of 465 nm using SoftMax Pro software on a Gemini EM instrument.

The total protein assay to be used was the Pierce 660 nm assay from Thermo Scientific. The 660 nm reagent is stored at room temperature. 10 μl of each standard was added in the wells of a microplate as well as 10 μl of the tissue lysates. The Protein Assay reagent was gently mixed and then 150 μl was added to both wells containing standards and samples.

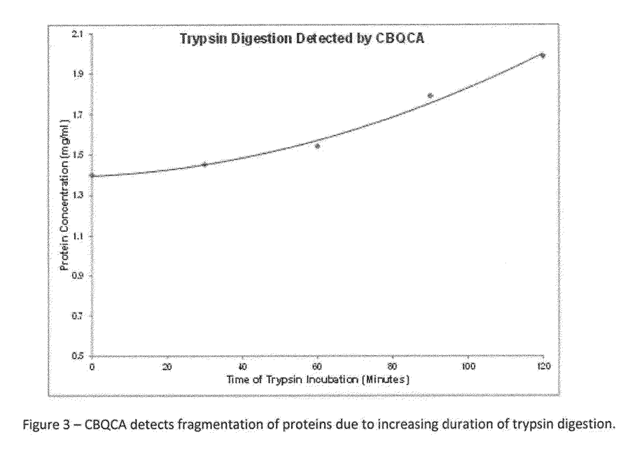

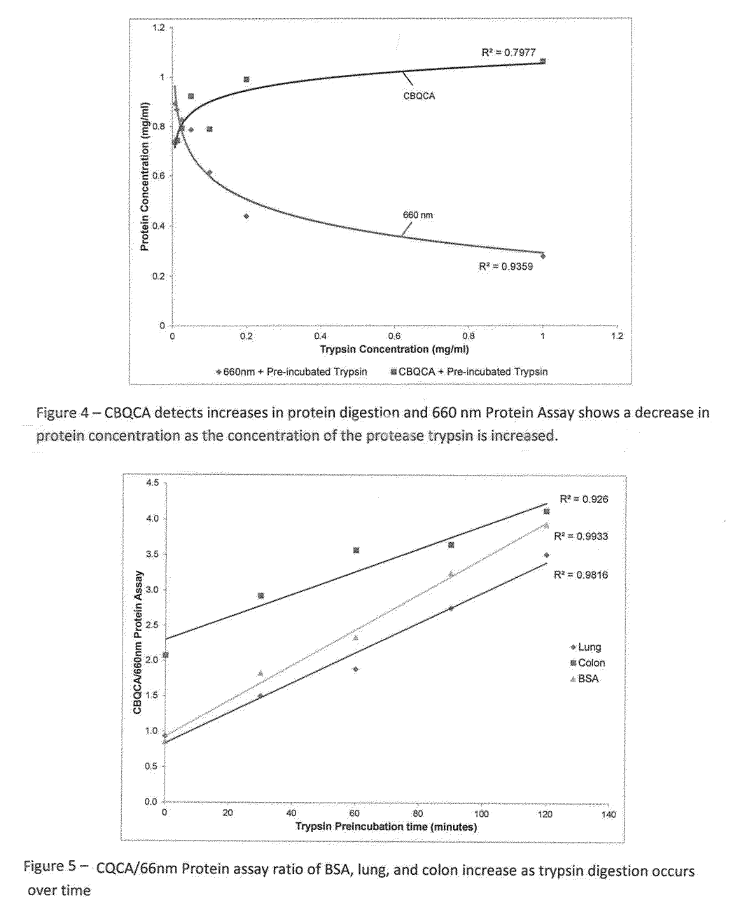

The assay with CBQCA detected an increase in free amine concentration indicating protein degradation (see FIG. 3). A decrease in protein content was measured with the 660 nm assay (see FIG. 4).

### EXAMPLE 5

Aliquots of freezer milled human lung and colon samples were weighed and suspended in 200 mM NaCl, 0.2% Triton pH 7.0. The samples were vortexed and sonicated. All samples were centrifuged in a SS34 rotor at 3000 RPM for 6 min. Upon completion of the centrifugation, samples were placed on ice. To prepare the standards for the protein assays, 150 μl of the BSA stock solution from Pierce (2 mg/ml) was placed in a microcentrifuge tube containing 50 μl of suspension solution (Final Conc. 1.5 mg/ml).

Trypsin was freshly prepared by adding 5.1 mg trypsin to 5.1 mL of 0.1 M sodium borate pH 9.3. 55 μl of the tissue lysates or BSA was added to 6 wells of an 8-well PCR strip. 2.2 μl of 0.1M sodium borate pH 9.3 was added to the lysate in the first well of each strip. 2.2 μl of trypsin was added to the one well of lysate and samples were placed at 37° C. Every 30 minutes, samples were removed and 2.2 μl of trypsin was added to a new lysate well. Samples were returned to 37° C. between each time point (30, 60, 90, 120 min.). Immediately prior to setting up the protein assays, 2.2 μl of trypsin was added to the last lysate well. Samples were kept on ice.

The Protein Degradation Assay was set up using CBQCA. To set up this assay, 31.8 μl of reaction buffer (0.1 M sodium borate pH 9.3) was added to each well of a black clear bottom 384 well plate. 2 μl of standards or sample were added to the well containing 0.1 M sodium borate. 1.25 μl of KCN was added to each well and mixed. A fresh 2 mM working stock of CBQCA was made by diluting the 40 mM stock solution 1:20 in 0.1 M sodium borate pH 9.3. 2.5 μl of the aqueous working solution were added immediately to each well as indicated. The samples were protected from light and mixed at room temperature for 1 hour. The plate was read at a 550 nm emission with an excitation of 465 nm using SoftMax Pro software on a Gemini EM instrument.

The total protein assay to be used was the Pierce 660 nm Protein Assay from Thermo Scientific. The 660 nm reagent is stored at room temperature. 10 μl of each standard was added to the wells in a microplate as well as 10 μl of the tissue lysates. The 660 nm protein assay reagent was gently mixed and then 150 μl was added to wells containing standards and samples.

The plates were shaken for 1 minute and then incubated at room temperature for 5 minutes. Then the plate was read on the SpectraMax reader using SoftMax Pro software at 660 nm absorbance.

As illustrated in FIG. 6, the CBQCA/660 nm Protein Assay ratio can be used to monitor protein degradation, e.g. increases with increased trypsin digestion. By employing both an assay that utilizes a fluorescent tag for free amines, e.g., 3-(4-carboxybenzoyl)quinoline-2-carboxaldehyde (CBQCA) and a standard protein assay, e.g. Bradford protein assay, BCA method (bicinchoninic acid), Lowry assay or the Pierce 660 nm Protein Assay, an even more sensitive assay can be provided as illustrated in FIG. 6. The ratio of the two assays increases as protein degradation increases.

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

All publications, patents and patent applications cited in this specification are herein incorporated by reference, and for any and all purpose, as if each individual publication, patent or patent application were specifically and individually indicated to be incorporated by reference. In the case of inconsistencies, the present disclosure will prevail.

The foregoing description of the disclosure illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The embodiments described hereinabove are further intended to explain best modes known of practicing it and to enable others skilled in the art to utilize the disclosure in such, or other, embodiments and with the various modifications required by the particular applications or uses. Accordingly, the description is not intended to limit it to the form disclosed herein. Also, it is intended that the appended claims be construed to include alternative embodiments.

What is claimed is:

1. An assay for determining the protein, polypeptide, and peptides degradation of a sample which comprises contacting the sample with a non-fluorescent compound whereby the non-fluorescent compound reacts with free primary amine in the sample from degradation of protein, polypeptide, and peptides in the sample producing a fluorescent signal to thereby measure the increase in protein, polypeptide, and peptides fragments in the sample and comparing the fluorescent signal to a standard to thereby quantify the amount of protein, polypeptide, and peptides degradation.

2. The assay according to claim 1, wherein the non-fluorescent compound is selected from the group consisting of 3-(4-carboxybenzoyl)quinoline-2-carboxaldehyde (CBQCA), fluorescamine and o-phthaldialdehyde.

3. The assay according to claim 1, wherein the non-fluorescent compound is 3-(4-carboxybenzoyl)quinoline-2-carboxaldehyde (CBQCA).

4. The assay according to claim 3, which further comprises adding a cyanide in the sample.

5. The assay according to claim 1, wherein the standard is from bovine serum albumin, bovine gamma globulin, or other purified proteins, polypeptides, or peptides.

6. The assay according to claim 1, which further comprises assaying the sample using a common protein assay.

7. The assay according to claim 6, wherein said standard protein assay is selected from the group consisting of Bradford protein assay, DC protein assay, BCA method, Lowry protein assay, and Pierce 660 nm protein assay.

8. The assay according to claim 7, wherein the standard is from bovine serum albumin, bovine gamma globulin, or other purified proteins or polypeptides.

9. The assay according to claim 6, wherein the ratio of the concentration free amines in a sample to the protein concentration in a sample is calculated to obtain a measure of protein, polypeptide, and peptide degradation.

10. The assay according to claim 9, wherein the non-fluorescent compound is selected from group consisting of 3-(4-carboxybenzoyl) quinoline-2-carboxaldehyde (CBQCA), fluorescamine and o-phthaldialdehyde.

11. The assay according to claim 7, wherein the non-fluorescent compound is 3-(4-carboxybenzoyl) quinoline-2-carboxaldehyde (CBQCA)

12. The assay according to claim 11, which further comprises adding a cyanide in the sample.

13. The assay according to claim 6, wherein the non-fluorescent compound is selected from the group consisting of 3-(4-carboxybenzoyl) quinoline-2-carboxaldehyde (CBQCA), fluorescamine and o-phthaldialdehyde.
